(19) United States
(12) Patent Application Publication
(10) Pub. No.: US 2021/0161897 A1
(43) Pub. Date: Jun. 3, 2021

(54) EPIDERMAL GROWTH FACTOR RECEPTOR TYROSINE KINASE INHIBITORS FOR THE TREATMENT OF CANCER

(71) Applicant: AstraZeneca AB, Södertälje (SE)

(72) Inventors: Nicolas Floch, Cambridge (GB); Paul David Smith, Cambridge (GB); Matthew Joseph Martin, Cambridge (GB)

(21) Appl. No.: 17/094,893

(22) Filed: Nov. 11, 2020

Related U.S. Application Data
(60) Provisional application No. 62/934,116, filed on Nov. 12, 2019.

(51) Int. Cl. A61K 31/517; A61P 35/00; A61K 31/506

(52) U.S. Cl. CPC A61K 31/517; A61K 31/506; A61P 35/00

(57) ABSTRACT

The specification relates to epidermal growth factor receptor (EGFR) tyrosine kinase inhibitors (TKIs) for use in the treatment of cancer, wherein the EGFR TKI is administered in combination with AZD2811.

### RELATED APPLICATION

This application claims benefit of priority under 35 U.S.C. § 119(e) of the U.S. Provisional Application No. 62/934,116, filed Nov. 12, 2019, which is incorporated by reference herein in its entirety for all purposes.

### FIELD

The specification relates to an Epidermal Growth Factor Receptor (EGFR) Tyrosine Kinase Inhibitor (TKI) for use in the treatment of cancer, wherein the EGFR TKI is administered in combination with AZD2811.

### BACKGROUND

The discovery of activating mutations in the epidermal growth factor receptor (EGFR) has revolutionized the treatment of the disease. In 2004, it was reported that activating mutations in exons 18-21 of EGFR correlated with a response to EGFR-TKI therapy in NSCLC (Science [2004], vol. 304, 1497-1500; New England Journal of Medicine [2004], vol. 350, 2129-2139). It is estimated that these mutations are prevalent in approximately 10-16% of NSCLC human patients in the United States and Europe, and in approximately 30-50% of NSCLC human patients in Asia. Two of the most significant EGFR activating mutations are the exon 19 deletions and the missense mutations in exon 21. The exon 19 deletions account for approximately 45% of known EGFR mutations. Eleven different mutations, resulting in deletion of three to seven amino acids, have been detected in exon 19, and all are centred around the uniformly deleted codons for amino acids 747-749. The most significant exon 19 deletion is E746-A750. The missense mutations in exon 21 account for approximately 39-45% of known EGFR mutations, of which the substitution mutation L858R accounts for approximately 39% of the total mutations in exon 21 (J. Thorac. Oncol. [2010], 1551-1558).

Two first generation (erlotinib & gefitinib), two second generation (afatinib & dacomitinib) and a third generation (osimertinib) epidermal growth factor receptor (EGFR) tyrosine kinase inhibitors (TKIs) are currently available for the management of EGFR mutation-positive NSCLC. All these TKIs are effective in patients with NSCLC whose tumour harbour the in-frame deletions in exon 19 and the L858R point mutation in exon 21. These two mutations represent approximatively 90% of all EGFR mutations. In approximately 50% of patients, resistance to first and second generation TKI is mediated by the acquisition of the ‘gatekeeper’ mutation T790M. Currently, osimertinib is the only registered EGFR TKI that is active against exon 19 deletions and L858R mutation, regardless of the presence of T790M mutation. However, even patients treated with osimertinib ultimately progress, predominantly due to the development of acquired resistance resulting from other resistance mechanisms. As such, there remains a need to develop new therapies for the treatment of NSCLC, especially for patients whose disease has progressed following treatment with a third generation EGFR TKI.

Aurora kinase exist in three classes (A, B and C) and Aurora kinase activation has been identified as a potential mechanism associated with acquired resistance to EGFR TKIs. Shah et al. (Nature Medicine, 2019, vol 25, page 111-118) identified Aurora kinase A (AURKA) as a relevant target in this setting. Bertran-Alamillo et al. (Nature Communications, 2019, 10(1):1812; https://doi.org/10.1038/s41467-019-09734-5) also reported that Aurora kinase activation is associated with acquired resistance to the first-generation EGFR TKIs erlotinib and gefitinib.

### SUMMARY

The present specification provides a means for overcoming acquired resistance to EGFR TKI treatment in NSCLC, utilising Aurora kinase B inhibitor AZD2811 in combination with EGFR TKIs.

Through laboratory experiments with populations of cancer cells resistant to osimertinib, it has been found that resistance to EGFR TKIs may be overcome in some patients by the use of AZD2811.

It has also been found that a combination of EGFR TKI and AZD2811 may provide an effective first-line therapy against EGFR-associated cancer, i.e. in patients who have not received previous treatment with an EGFR TKI (referred to herein as EGFR TKI-naïve patients). In such patients, the combination treatment may act to delay or prevent development of resistance.

Furthermore, it has been found that the subset of cells that survive EGFR TKI treatment but exist in a non-proliferative pre-resistant state (herein referred to as Drug Tolerant Persister (DTP) cells) are sensitive to AZD2811, and that treatment with AZD2811 resulted in cell death.

Without being bound by theory, it is proposed that, in cells addicted to the EGFR pathway, inhibition of this pathway induces a state in which cells are susceptible to AZD2811. Cells chronically treated with EGFR TKI monotherapy find alternative mechanisms to circumvent EGFR inhibition, (e.g. by activating bypass signalling pathways), which enables the cells to survive in the absence of EGFR signalling, and so allows disease progression in patients. However, in a subset of these patients, cellular adaptations required for proliferation in the presence of EGFR inhibition may uncover a novel vulnerability to Aurora kinase B inhibition. In preclinical cell line models, a subset of cells resistant to osimertinib showed enhanced sensitivity to AZD2811 compared to osimertinib-sensitive parental cells, either in the absence or presence of co-dosed osimertinib. A smaller subset of these cells showed enhanced sensitivity when osimertinib was co-dosed. Resistant cells which display enhanced sensitivity to AZD2811 demonstrated an accelerated entry into the S-phase of the cell cycle (after synchronization) compared to parental cells. Therefore, a high proliferative index (i.e. a high level of cell division) in patient tumour tissue may be a potential biomarker for sensitivity to AZD2811 in patients.

It was further found that cells that were tolerant to osimertinib but that did not proliferate in the presence of osimertinib had increased sensitivity to AZD2811 compared to parental cell lines when AZD2811 was given as a monotherapy. AZD2811 induced a modest but significant level of apoptosis in DTP cells at doses that did not affect parental cells. Accordingly, DTP cells induced by EGFR-inhibitor treatment may exist in a cell cycle context that makes them sensitive to AZD2811.

This specification thus shows the potential for a combination of an EGFR TKI and AZD2811 to demonstrate efficacy both as a first-line treatment (i.e. in EGFR TKI-naïve patients) and as a second-line treatment (i.e. in patients previously treated with EGFR TKIs, including patients whose disease has progressed on or after such treatment) of EGFR-mutant NSCLC.

In a first aspect, there is provided an EGFR TKI for use in the treatment of cancer in a human patient, wherein the EGFR TKI is administered in combination with AZD2811.

In a further aspect, there is provided a method of treating cancer in a human patient in need of such a treatment comprising administration to the human patient a therapeutically effective amount of an EGFR TKI, wherein the EGFR TKI is administered in combination with a therapeutically effective amount of AZD2811.

In a further aspect, there is provided the use of an EGFR TKI in the manufacture of a medicament for the treatment of cancer in a human patient, wherein the EGFR TKI is administered in combination with AZD2811.

### DESCRIPTION OF FIGURES

FIG. 1: A subset of osimertinib-resistant cell lines show enhanced sensitivity to Aurora Kinase B inhibitors. Dose response curves were generated to determine an IC50 for AZD2811 in resistant and parental PC9 cell lines.

FIG. 2: A subset of osimertinib-resistant cell lines show enhanced sensitivity to Aurora Kinase B inhibitors in combination with osimertinib (“OSI”). Dose response curves were generated to determine an IC50 for AZD2811 in resistant and parental PC9 cell lines.

FIG. 3: Osimertinib-resistant cell lines that show enhanced sensitivity to Aurora Kinase B inhibition undergo apoptosis in response to drug. Caspase activity assays were used in both parental and an osimertinib-resistant cell line at various doses of AZD2811.

FIG. 4: Osimertinib DTPs are sensitive to Aurora Kinase B inhibition. Parental PC9 cells were treated with the combination of osimertinib and AZD2811 to determine the rate of DTP formation. Cell confluence was measured on the Incucyte imaging platform as a surrogate for DTP number.

FIG. 5: Osimertinib DTPs are sensitive to Aurora Kinase B inhibition. Parental PC9 cells were treated with osimertinib monotherapy to generate DTPs and then treated with AZD2811. Cell confluence was measured on the Incucyte imaging platform as a surrogate for DTP number.

FIG. 6: Aurora Kinase B inhibition induces apoptosis in DTPs and in a subset of parental cells when given in combination with osimertinib. DTPs were generated by treatment with osimertinib monotherapy for 14 days, followed by treatment with osimertinib in combination with AZD2811 for 72 h. Cells were co-treated with a green fluorescent caspase activity reagent and monitored over time on the Incucyte imaging platform.

FIG. 7: Aurora Kinase B inhibition induces apoptosis in DTPs and in a subset of parental cells when given in combination with osimertinib. Similarly, a panel of osimertinib sensitive cell lines were treated with the combination of osimertinib and AZD2811 (10, 30 or 100 nM) an monitored for caspase activity on the Incucyte. The plotted ratio represents the level of caspase activation at 48 h compared to that induced by osimertinib treatment alone.

FIG. 8: AZD2811 combined with osimertinib prevents the regrowth of PC9 xenograft compared to osimertinib monotherapy. In vivo antitumour efficacy following 4 weeks of daily treatment of vehicle, osimertinib 25 mg/kg, once weekly AZD2811 or the combination of osimertinib and AZD2811. Data are represented as mean±SEM (n=5 per group).

### DETAILED DESCRIPTION

### EGFR Mutation Positive NSCLC and Diagnostic Methods

In embodiments, the cancer is lung cancer, such as non-small cell lung cancer (NSCLC).

In embodiments, the NSCLC is an EGFR mutation-positive NSCLC.

In embodiments, the EGFR mutation-positive NSCLC comprises activating mutations in EGFR. In further embodiments, the EGFR mutation-positive NSCLC comprises non-resistant mutations. In further embodiments, the activating mutations in EGFR comprise activating mutations in exons 18-21. In further embodiments, the activating mutations in EGFR comprise exon 19 deletions or missense mutations in exon 21. In further embodiments, the activating mutations in EGFR comprise exon 19 deletions or L858R substitution mutations. In further embodiments, the mutations in EGFR comprise the T790M mutation.

In embodiments, the EGFR mutation-positive NSCLC is a locally-advanced EGFR mutation-positive NSCLC.

In embodiments, the EGFR mutation-positive NSCLC is a metastatic EGFR mutation-positive NSCLC.

In embodiments, the EGFR mutation-positive NSCLC is not amenable to curative surgery or radiotherapy.

There are numerous methods to detect EGFR activating mutations, of which the skilled person will be aware. A number of tests suitable for use in these methods have been approved by the US Food and Drug Administration (FDA). These include both tumour tissue and plasma based diagnostic methods. In general, the EGFR mutation status is first assessed using a tumour tissue biopsy sample derived from the human patient. If a tumour sample is unavailable, or if the tumour sample is negative, the EGFR mutation status may be assessed using a plasma sample. A particular example of a suitable diagnostic test to detect EGFR mutations, and in particular to detect exon 19 deletions, L858R substitution mutations and the T790M mutation, is the Cobas™ EGFR Mutation Test v2 (Roche Molecular Diagnostics).

In embodiments, therefore, the EGFR mutation-positive NSCLC comprises activating mutations in EGFR (such as activating mutations in exons 18-21, for example exon 19 deletions, missense mutations in exon 21, and L858R substitution mutations; and resistance mutations such as the T790M mutation), wherein the EGFR mutation status of the human patient has been determined using an appropriate diagnostic test. In further embodiments, the EGFR mutation status has been determined using a tumour tissue sample. In further embodiments, the EGFR mutation status has been determined using a plasma sample.

In further embodiments, the diagnostic method uses an FDA-approved test. In further embodiments, the diagnostic method uses the Cobas™ EGFR Mutation Test (v1 or v2).

In embodiments, the human patient is an EGFR TKI-naïve human patient.

In embodiments the human patient has previously received EGFR TKI treatment. In embodiments the human patient has previously been treated with osimertinib, or a pharmaceutically acceptable salt thereof. In further embodiments, the human patient's disease has progressed on or after previous EGFR TKI treatment. In further embodiments, the human patient's disease has progressed on or after previous treatment with osimertinib, or a pharmaceutically acceptable salt thereof. EGFR TKI treatment includes treatment with either a first-, second- or third-generation EGFR TKI or combinations thereof. In embodiments, the human patient has developed EGFR T790M mutation-positive NSCLC.

In embodiments, the administration of EGFR TKI in combination with AZD2811 induces cell death in drug tolerant persister cells.

### EGFR TKIs

EGFR TKIs can be characterised as either first-, second- or third-generation EGFR TKIs, as set out below.

First-generation EGFR TKIs are reversible inhibitors of EGFR bearing activating mutations that do not significantly inhibit EGFR bearing a T790M mutation. Examples of first-generation TKIs include gefitinib and erlotinib.

Second-generation EGFR TKIs are irreversible inhibitors of EGFR bearing activating mutations that do not significantly inhibit EGFR bearing the T790M mutation. Examples of second-generation TKIs include afatinib and dacomitinib.

Third-generation EGFR TKIs are inhibitors of EGFR bearing activating mutations that also significantly inhibit EGFR bearing the T790M mutation and do not significantly inhibit wild-type EGFR. Examples of third-generation TKIs include compounds of Formula (I), osimertinib, AZD3759, lazertinib, nazartinib, C01686 (rociletinib), HM61713, ASP8273, EGF816, PF-06747775 (mavelertinib), avitinib (abivertinib), alflutinib (AST2818) and CX-101 (RX-518), almonertinib (HS-10296) and BPI-7711.

In embodiments, the EGFR TKI is a first-generation EGFR TKI. In further embodiments, the first-generation EGFR TKI is selected from the group consisting of gefitinib or a pharmaceutically acceptable salt thereof, icotinib or a pharmaceutically acceptable salt thereof, and erlotinib or a pharmaceutically acceptable salt thereof.

In embodiments, the EGFR TKI is a second-generation EGFR TKI. In further embodiments, the second-generation EGFR TKI is selected from dacomitinib, or a pharmaceutically acceptable salt thereof and afatinib, or a pharmaceutically acceptable salt thereof.

In embodiments, the EGFR TKI is a third-generation EGFR TKI. In further embodiments, the third-generation EGFR TKI is a compound of Formula (I), as defined below. In further embodiments, the third-generation EGFR TKI is selected from the group consisting of osimertinib or a pharmaceutically acceptable salt thereof, AZD3759 or a pharmaceutically acceptable salt thereof, lazertinib or a pharmaceutically acceptable salt thereof, abivertinib or a pharmaceutically acceptable salt thereof, alflutinib or a pharmaceutically acceptable salt thereof, CX-101 or a pharmaceutically acceptable salt thereof, HS-10296 or a pharmaceutically acceptable salt thereof and BPI-7711 or a pharmaceutically acceptable salt thereof. In further embodiments, the third generation EGFR TKI is osimertinib or a pharmaceutically acceptable salt thereof.

### Compounds of Formula (I)

In an embodiment, the EGFR TKI is a compound of Formula (I):

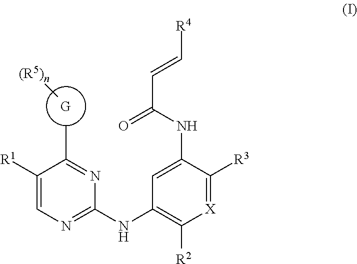

wherein:

G is selected from 4,5,6,7-tetrahydropyrazolo[1,5-a]pyridin-3-yl, indol-3-yl, indazol-1-yl, 3,4-dihydro-1H-[1,4]oxazino[4,3-a]indol-10-yl, 6,7,8,9-tetrahydropyrido[1,2-a]indol-10-yl, 5,6-dihydro-4H-pyrrolo[3,2,1-ij]quinolin-1-yl, pyrrolo[3,2-b]pyridin-3-yl and pyrazolo[1,5-a]pyridin-3-yl;

R¹ is selected from hydrogen, fluoro, chloro, methyl and cyano;

R² s selected from methoxy, trifluoromethoxy, ethoxy, 2,2,2-trifluoroethoxy and methyl;

R³ is selected from (3R)-3-(dimethylamino)pyrrolidin-1-yl, (3S)-3-(dimethyl-amino)pyrrolidin-1-yl, 3-(dimethylamino)azetidin-1-yl, [2-(dimethylamino)ethyl]-(methyl)amino, [2-(methylamino)ethyl](methyl)amino, 2-(dimethylamino)ethoxy, 2-(methylamino)ethoxy, 5-methyl-2,5-diazaspiro[3.4]oct-2-yl, (3aR,6aR)-5-methylhexa-hydro-pyrrolo[3,4-b]pyrrol-1(2H)-yl, 1-methyl-1,2,3,6-tetrahydropyridin-4-yl, 4-methylpiperizin-1-yl, 4-[2-(dimethylamino)-2-oxoethyl]piperazin-1-yl, methyl[2-(4-methylpiperazin-1-yl)ethyl]amino, methyl[2-(morpholin-4-yl)ethyl]amino, 1-amino-1,2,3,6-tetrahydropyridin-4-yl and 4-[(2S)-2-aminopropanoyl]piperazin-1-yl;

R⁴ is selected from hydrogen, 1-piperidinomethyl and N,N-dimethylaminomethyl;

R⁵ is independently selected from methyl, ethyl, propyl, 2,2-difluoroethyl, 2,2,2-trifluoroethyl, fluoro, chloro and cyclopropyl;

X is CH or N; and

n is 0, 1 or 2;

or a pharmaceutically acceptable salt thereof.

In a further embodiment there is provided a compound of Formula (I), as defined above, wherein G is selected from indol-3-yl and indazol-1-yl; R¹ is selected from hydrogen, fluoro, chloro, methyl and cyano; R² is selected from methoxy and 2,2,2-trifluoroethoxy; R³ is selected from [2-(dimethylamino)ethyl]-(methyl)amino, [2-(methylamino)ethyl](methyl)amino, 2-(dimethylamino)ethoxy and 2-(methylamino)ethoxy; R⁴ is hydrogen; R⁵ is selected from methyl, 2,2,2-trifluoroethyl and cyclopropyl; X is CH or N; and n is 0 or 1; or a pharmaceutically acceptable salt thereof.

Examples of compounds of Formula (I) include those described in WO 2013/014448, WO 2015/175632, WO 2016/054987, WO 2016/015453, WO 2016/094821, WO 2016/070816 and WO 2016/173438.

### Osimertinib and Pharmaceutical Compositions Thereof

Osimertinib has the following chemical structure:

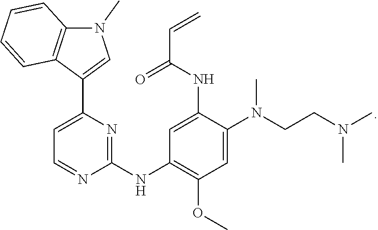 [SMILES: C=CC(=O)Nc1cc(Nc2nccc(-c3cn(C)c4ccccc34)n2)c(OC)cc1N(C)CCN(C)C]

The free base of osimertinib is known by the chemical name: N-(2-{2-dimethylamino ethyl-methylamino}-4-methoxy-5-{[4-(1-methylindol-3-yl)pyrimidin-2-yl]amino}phenyl) prop-2-enamide. Osimertinib is described in WO 2013/014448. Osimertinib is also known as AZD9291.

Osimertinib may be found in the form of the mesylate salt: N-(2-{2-dimethylamino ethyl-methylamino}-4-methoxy-5-{[4-(1-methylindol-3-yl)pyrimidin-2-yl]amino}phenyl) prop-2-enamide mesylate salt. Osimertinib mesylate is also known as TAGRISSO™.

Osimertinib mesylate is currently approved as an oral once daily tablet formulation, at a dose of 80 mg (expressed as free base, equivalent to 95.4 mg osimertinib mesylate), for the treatment of metastatic EGFR T790M mutation positive NSCLC human patient s. A 40 mg oral once daily tablet formulation (expressed as free base, equivalent to 47.7 mg osimertinib mesylate) is available should dose modification be required. The tablet core comprises pharmaceutical diluents (such as mannitol and microcrystalline cellulose), disintegrants (such as low-substituted hydroxypropyl cellulose) and lubricants (such as sodium stearyl fumarate). The tablet formulation is described in WO 2015/101791.

In embodiments, therefore, osimertinib, or a pharmaceutically acceptable salt thereof, is in the form of the mesylate salt, i.e. N-(2-{2-dimethylamino ethyl-methylamino}-4-methoxy-5-{[4-(1-methylindol-3-yl)pyrimidin-2-yl]amino}phenyl) prop-2-enamide mesylate salt.

In embodiments, osimertinib, or a pharmaceutically acceptable salt thereof, is administered once-daily. In further embodiments, osimertinib mesylate is administered once-daily.

In embodiments, the total daily dose of osimertinib is about 80 mg. In further embodiments, the total daily dose of osimertinib mesylate is about 95.4 mg.

In embodiments, the total daily dose of osimertinib is about 40 mg. In further embodiments, the total daily dose of osimertinib mesylate is about 47.7 mg.

In embodiments, osimertinib, or a pharmaceutically acceptable salt thereof, is in tablet form.

In embodiments, osimertinib, or a pharmaceutically acceptable salt thereof, is administered in the form of a pharmaceutical composition comprising one or more pharmaceutically acceptable excipients. In further embodiments, the composition comprises one or more pharmaceutical diluents (such as mannitol and microcrystalline cellulose), one or more pharmaceutical disintegrants (such as low-substituted hydroxypropyl cellulose) or one or more pharmaceutical lubricants (such as sodium stearyl fumarate).

In embodiments, the composition is in the form of a tablet, wherein the tablet core comprises: (a) from 2 to 70 parts of osimertinib, or a pharmaceutically acceptable salt thereof; (b) from 5 to 96 parts of two or more pharmaceutical diluents; (c) from 2 to 15 parts of one or more pharmaceutical disintegrants; and (d) from 0.5 to 3 parts of one or more pharmaceutical lubricants; and wherein all parts are by weight and the sum of the parts (a)+(b)+(c)+(d)=100.

In embodiments, the composition is in the form of a tablet, wherein the tablet core comprises: (a) from 7 to 25 parts of osimertinib, or a pharmaceutically acceptable salt thereof; (b) from 55 to 85 parts of two or more pharmaceutical diluents, wherein the pharmaceutical diluents comprise microcrystalline cellulose and mannitol; (c) from 2 to 8 parts of pharmaceutical disintegrant, wherein the pharmaceutical disintegrant comprises low-substituted hydroxypropyl cellulose; (d) from 1.5 to 2.5 parts of pharmaceutical lubricant, wherein the pharmaceutical lubricant comprises sodium stearyl fumarate; and wherein all parts are by weight and the sum of the parts (a)+(b)+(c)+(d)=100.

In embodiments, the composition is in the form of a tablet, wherein the tablet core comprises: (a) about 19 parts of osimertinib mesylate; (b) about 59 parts of mannitol; (c) about 15 parts of microcrystalline cellulose; (d) about 5 parts of low-substituted hydroxypropyl cellulose; and (e) about 2 parts of sodium stearyl fumarate; and wherein all parts are by weight and the sum of the parts (a)+(b)+(c)+(d)+(e)=100.

### AZD3759

AZD3759 has the following chemical structure:

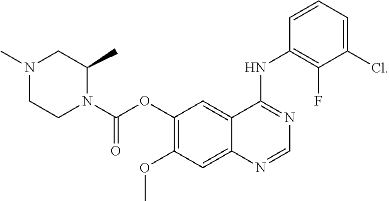 [SMILES: COc1cc2ncnc(Nc3cccc(Cl)c3F)c2cc1OC(=O)N1CCN(C)C[C@H]1C]

The free base of AZD3759 is known by the chemical name: 4-[(3-chloro-2-fluorophenyl)amino]-7-methoxy-6-quinazolinyl (2R)-2,4-dimethyl-1-piperazinecarboxylate. AZD3759 is disclosed in WO 2014/135876.

In embodiments, AZD3759, or a pharmaceutically acceptable salt thereof, is administered twice-daily. In further embodiments, AZD3759 is administered twice-daily.

In embodiments, the total daily dose of AZD3759 is about 400 mg. In further embodiments, about 200 mg of AZD3759 is administered twice a day.

### Lazertinib

Lazertinib has the following chemical structure:

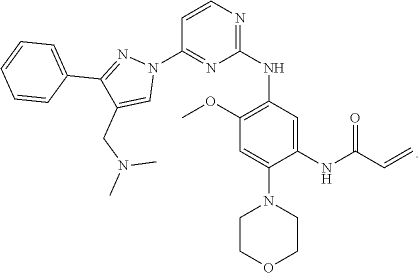 [SMILES: C=CC(=O)Nc1cc(Nc2nccc(-n3cc(CN(C)C)c(-c4ccccc4)n3)n2)c(OC)cc1N1CCOCC1]

The free base of lazertinib is known by the chemical name: N-{5-[(4-{4-[(dimethylamino)methyl]-3-phenyl-1H-pyrazol-1-yl}-2-pyrimidinyl)amino]-4-methoxy-2-(4-morpholinyl)phenyl}acrylamide. Lazertinib is described in WO 2016/060443. Lazertinib is also known by the names YH25448 and GNS-1480.

In embodiments, lazertinib, or a pharmaceutically acceptable salt thereof, is administered once-daily. In further embodiments, lazertinib is administered once-daily.

In embodiments, the total daily dose of lazertinib is about 20 to 320 mg.

In embodiments, the total daily dose of lazertinib is about 240 mg.

### Avitinib

Avitinib has the following chemical structure:

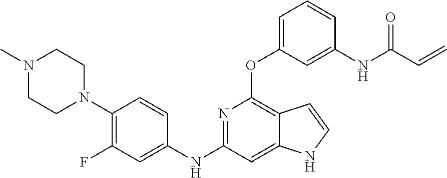 [SMILES: C=CC(=O)Nc1cccc(Oc2nc(Nc3ccc(N4CCN(C)CC4)c(F)c3)cc3[nH]ccc23)c1]

The free base of avitinib is known by the chemical name: N-(3-((2-((3-fluoro-4-(4-methylpiperazin-1-yl)phenyl)amino)-7H-pyrrolo(2,3-d)pyrimidin-4-yl)oxy)phenyl)prop-2-enamide. Avitinib is disclosed in US2014038940. Avitinib is also known as abivertinib.

In embodiments, avitinib, or a pharmaceutically acceptable salt thereof, is administered twice-daily. In further embodiments, avitinib maleate is administered twice-daily.

In embodiments, the total daily dose of avitinib maleate is about 600 mg.

### Alflutinib

Alflutinib has the following chemical structure:

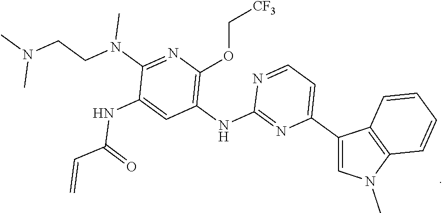 [SMILES: C=CC(=O)Nc1cc(Nc2nccc(-c3cn(C)c4ccccc34)n2)c(OCC(F)(F)F)nc1N(C)CCN(C)C]

The free base of alflutinib is known by the chemical name: N-(2-{[2-(dimethylamino)ethyl](methyl)amino}-6-(2,2,2-trifluoroethoxyl)-5-{[4-(1-methyl-1H-indol-3-yl)pyrimidin-2-yl]amino}pyridin-3-yl)acrylamide. Alflutinib is disclosed in WO 2016/15453. Alflutinib is also known as AST2818.

In embodiments, alflutinib, or a pharmaceutically acceptable salt thereof, is administered once-daily. In further embodiments, alflutinib mesylate is administered once-daily.

In embodiments, the total daily dose of alflutinib mesylate is about 80 mg.

In embodiments, the total daily dose of alflutinib mesylate is about 40 mg.

### Afatinib

Afatinib has the following chemical structure:

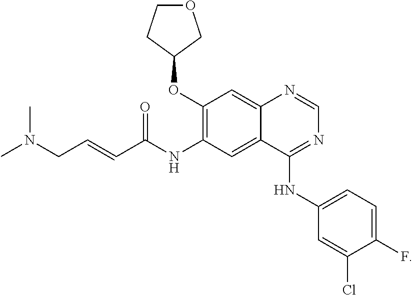 [SMILES: CN(C)C/C=C/C(=O)Nc1cc2c(Nc3ccc(F)c(Cl)c3)ncnc2cc1O[C@H]1CCOC1]

The free base of afatinib is known by the chemical name: N-[4-(3-chloro-4-fluoroanilino)-7-[(3)-oxolan-3-yl]oxyquinazolin-6-yl]-4-(dimethylamino)but-2-enamide. Afatinib is disclosed in WO 02/50043. Afatinib is also known as Gilotrif.

In embodiments, afatinib, or a pharmaceutically acceptable salt thereof, is administered once-daily. In further embodiments, afatinib dimaleate is administered once-daily.

In embodiments, the total daily dose of afatinib dimaleate is about 40 mg.

In embodiments, the total daily dose of afatinib dimaleate is about 30 mg.

### CX-101

CX-101 has the following chemical structure:

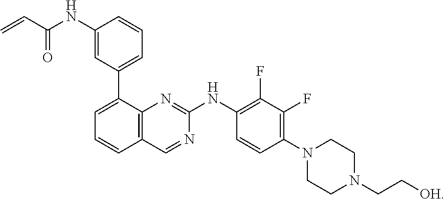 [SMILES: C=CC(=O)Nc1cccc(-c2cccc3cnc(Nc4ccc(N5CCN(CCO)CC5)c(F)c4F)nc23)c1]

The free base of CX-101 is known by the chemical name: N-(3-(2-((2,3-difluoro-4-(4-(2-hydroxyethyl)piperazin-1-yl)phenyl)amino)quinazolin-8-yl)phenyl)acrylamide. CX-101 is disclosed in WO 2015/027222. CX-101 is also known as RX-518.

### HS-10296 (Almonertinib)

HS-10296 (almonertinib) has the following chemical structure:

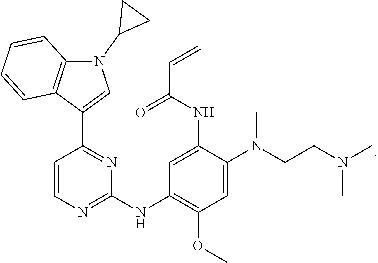 [SMILES: C=CC(=O)Nc1cc(Nc2nccc(-c3cn(C4CC4)c4ccccc34)n2)c(OC)cc1N(C)CCN(C)C]

The free base of HS-10296 is known by the chemical name: N-[5-[[4-(1-cyclopropylindol-3-yl)pyrimidin-2-yl]amino]-2-[2-(dimethylamino)ethyl-methyl-amino]-4-methoxy-phenyl]prop-2-enamide. HS-10296 is disclosed in WO 2016/054987.

In embodiments, the total daily dose of HS-10296 is about 110 mg.

### Icotinib

Icotinib has the following chemical structure:

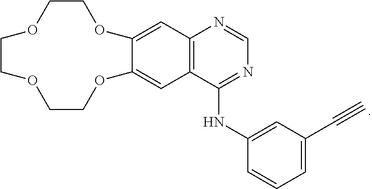 [SMILES: C#Cc1cccc(Nc2ncnc3cc4c(cc23)OCCOCCOCCO4)c1]

The free base of icotinib is known by the chemical name: N-(3-ethynylphenyl)-2,5,8,11-tetraoxa-15,17-diazatricyclo[10.8.0.0^{14,19}]icosa-1(12),13,15,17,19-pentaen-18-amine. Icotinib is disclosed in WO2013064128. Icotinib is also known as Conmana.

In embodiments, icotinib, or a pharmaceutically acceptable salt thereof, is administered three times daily. In further embodiments, icotinib hydrochloride is administered three times daily.

In embodiments, the total daily dose of icotinib hydrochloride is about 375 mg.

### BPI-7711

BPI-7711 has the following chemical structure:

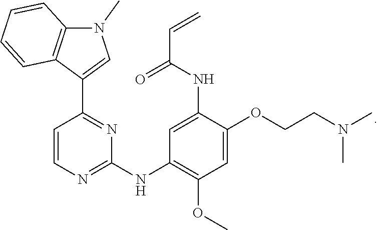 [SMILES: C=CC(=O)Nc1cc(Nc2nccc(-c3cn(C)c4ccccc34)n2)c(OC)cc1OCCN(C)C]

The free base of BPI-7711 is known by the chemical name: N-[2-[2-(dimethylamino)ethoxy]-4-methoxy-5-[[4-(1-methylindol-3-yl)pyrimidin-2-yl]amino]phenyl]prop-2-enamide. BPI-7711 is disclosed in WO 2016/94821.

In embodiments, the total daily dose of BPI-7711 is about 180 mg.

### Dacomitinib

Dacomitinib has the following chemical structure:

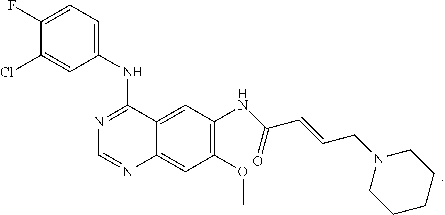 [SMILES: COc1cc2ncnc(Nc3ccc(F)c(Cl)c3)c2cc1NC(=O)/C=C/CN1CCCCC1]

The free form of dacomitinib is known by the chemical name: (2E)-N-{4-[(3-chloro-4-fluorophenyl)amino]-7-methoxyquinazolin-6-yl}-4-(piperidin-1-yl)but-2-enamide. Dacomitinib is disclosed in WO 2005/107758. Dacomitinib is also known by the name PF-00299804.

Dacomitinib may be found in the form of dacomitinib monohydrate, i.e. (2E)-N-{4-[(3-chloro-4-fluorophenyl)amino]-7-methoxyquinazolin-6-yl}-4-(piperidin-1-yl)but-2-enamide monohydrate.

In embodiments, dacomitinib, or a pharmaceutically acceptable salt thereof, is administered once-daily. In further embodiments, dacomitinib monohydrate is administered once-daily.

In embodiments, the total daily dose of dacomitinib monohydrate is about 45 mg.

In embodiments, dacomitinib, or a pharmaceutically acceptable salt thereof, is in tablet form.

In embodiments, dacomitinib, or a pharmaceutically acceptable salt thereof, is administered in the form of a pharmaceutical composition comprising one or more pharmaceutically acceptable excipients. In further embodiments, the one or more pharmaceutically acceptable excipients comprise lactose monohydrate, microcrystalline cellulose, sodium starch glycolate and magnesium stearate.

### Gefitinib

Gefitinib has the following chemical structure:

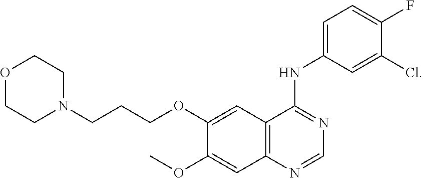 [SMILES: COc1cc2ncnc(Nc3ccc(F)c(Cl)c3)c2cc1OCCCN1CCOCC1]

The free base of gefitinib is known by the chemical name: N-(3-chloro-4-fluorophenyl)-7-methoxy-6-(3-morpholin-4-ylpropoxy)quinazolin-4-amine. Gefitinib is disclosed in WO 1996/033980. Gefitinib is also known as IRESSA™.

In embodiments, gefitinib, or a pharmaceutically acceptable salt thereof, is administered once-daily. In further embodiments, gefitinib is administered once-daily.

In embodiments, the total daily dose of gefitinib is about 250 mg.

### Erlotinib

Erlotinib has the following chemical structure:

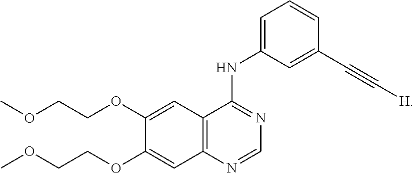

The free base of erlotinib is known by the chemical name: N-(3-ethynylphenyl)-6,7-bis(2-methoxyethoxy) quinazolin-4-amine. Erlotinib is disclosed in WO 1996/030347. Erlotinib is also known as TARCEVA™.

In embodiments, erlotinib, or a pharmaceutically acceptable salt thereof, is administered once-daily. In further embodiments, erlotinib is administered once-daily.

In embodiments, the total daily dose of erlotinib is about 150 mg.

In embodiments, the total daily dose of erlotinib is about 100 mg.

### AZD2811

The term “AZD2811” refers to a nanoparticle comprising between about 15 to about 25 weight percent of 2-(3-((7-(3-(ethyl(2-hydroxyethyl)amino)propoxy)quinazolin-4-yl)amino)-1H-pyrazol-5-yl)-N-(3-fluorophenyl)acetamide (also known as AZD1152 hqpa):

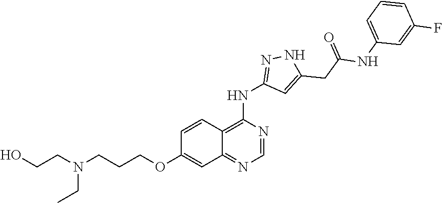 [SMILES: CCN(CCO)CCCOc1ccc2c(Nc3cc(CC(=O)Nc4cccc(F)c4)[nH]n3)ncnc2c1]

between about 7 to about 15 weight percent of pamoic acid, and a diblock poly(lactic) acid-poly(ethylene)glycol copolymer; wherein the diblock poly(lactic) acid-poly(ethylene)glycol copolymer has a poly(lactic acid) block having a number average molecular weight of about 16 kDa and a poly(ethylene)glycol block having a number average molecular weight of about 5 kDa; wherein the poly(ethylene)glycol block comprises about 10 to 30 weight percent of the therapeutic nanoparticle. In some embodiments, the nanoparticle comprises about 20 weight percent of 2-(3-((7-(3-(ethyl(2-hydroxyethyl)amino)propoxy)quinazolin-4-yl)amino)-1H-pyrazol-5-yl)-N-(3-fluorophenyl)acetamide.

Therapeutic nanoparticles comprising AZD1152 hqpa are disclosed in WO 2015/036792. AZD1152 hqpa is disclosed in WO 2004/058781.

In embodiments, the total daily dose of AZD2811 is about 200 mg via intravenous administration. In further embodiments, AZD2811 is administered on day 1 and day 4 of a 28-day cycle.

### FURTHER EMBODIMENTS

In an aspect there is provided an EGFR TKI for use in the treatment of cancer in a human patient, wherein the EGFR TKI is administered in combination with AZD2811. In embodiments, the cancer is lung cancer, such as NSCLC. In yet further embodiments, the NSCLC is an EGFR mutation-positive NSCLC.

In an aspect there is provided AZD2811 for use in the treatment of cancer in a human patient, wherein the AZD2811 is administered in combination with an EGFR TKI. In embodiments, the cancer is lung cancer, such as NSCLC. In yet further embodiments, the NSCLC is an EGFR mutation-positive NSCLC.

In an aspect there is provided a method of treating cancer in a human patient in need of such a treatment comprising the administration to the human patient of a therapeutically effective amount of an EGFR TKI, wherein the EGFR TKI is administered in combination with a therapeutically effective amount of AZD2811. In embodiments, the cancer is lung cancer, such as NSCLC. In yet further embodiments, the NSCLC is an EGFR mutation-positive NSCLC.

In an aspect there is provided the use of an EGFR TKI in the manufacture of a medicament for the treatment of cancer in a human patient, wherein the EGFR TKI is administered in combination with AZD2811. In embodiments, the cancer is lung cancer, such as NSCLC. In yet further embodiments, the NSCLC is an EGFR mutation-positive NSCLC.

In an aspect there is provided a combination of an EGFR TKI and AZD2811 for use in the treatment of cancer in a human patient. In embodiments the EGFR TKI is osimertinib or a pharmaceutically acceptable salt thereof. In further embodiments, the human patient is an EGFR TKI-naïve human patient. In further embodiments, the human patient has previously received EGFR TKI treatment. In further embodiments, the human patient has previously received osimertinib or a pharmaceutically acceptable salt thereof. In still further embodiments, the cancer is lung cancer, such as NSCLC. In yet further embodiments, the NSCLC is an EGFR mutation-positive NSCLC.

In an aspect there is provided a method of treating cancer in a human patient in need of such a treatment comprising administration to the human patient a combination of a therapeutically effective amount of an EGFR TKI and a therapeutically effective amount of AZD2811. In embodiments, the EGFR TKI is osimertinib or a pharmaceutically acceptable salt thereof. In further embodiments, the human patient is an EGFR TKI-naïve human patient. In further embodiments, the human patient has previously received EGFR TKI treatment. In further embodiments, the human patient has previously received osimertinib or a pharmaceutically acceptable salt thereof. In still further embodiments, the cancer is lung cancer, such as NSCLC. In yet further embodiments, the NSCLC is an EGFR mutation-positive NSCLC.

In an aspect there is provided the use of a combination of an EGFR TKI and AZD2811 in the manufacture of a medicament for treatment of cancer in a human patient. In embodiments, the EGFR TKI is osimertinib or a pharmaceutically acceptable salt thereof. In further embodiments, the human patient is an EGFR TKI-naïve human patient. In further embodiments, the human patient has previously received EGFR TKI treatment. In further embodiments, the human patient has previously received osimertinib or a pharmaceutically acceptable salt thereof. In still further embodiments, the cancer is lung cancer, such as NSCLC. In yet further embodiments, the NSCLC is an EGFR mutation-positive NSCLC.

In an aspect there is provided a combination of osimertinib or a pharmaceutically acceptable salt thereof and AZD2811 for use in the treatment of cancer in a human patient, wherein the osimertinib, or pharmaceutically acceptable salt thereof, is administered to the human patient before the AZD2811 is administered to the human patient. In embodiments, the cancer is lung cancer, such as NSCLC. In yet further embodiments, the NSCLC is an EGFR mutation-positive NSCLC.

In an aspect there is provided a method of treating cancer in a human patient in need of such a treatment comprising administration to the human patient a combination of a therapeutically effective amount of osimertinib or a pharmaceutically acceptable salt thereof and a therapeutically effective amount of AZD2811, wherein the osimertinib, or pharmaceutically acceptable salt thereof, is administered to the human patient before the AZD2811 is administered to the human patient. In embodiments, the cancer is lung cancer, such as NSCLC. In yet further embodiments, the NSCLC is an EGFR mutation-positive NSCLC.

In an aspect there is provided the use of a combination of osimertinib or a pharmaceutically acceptable salt thereof and AZD2811 for the manufacture of a medicament for the treatment of cancer in a human patient, wherein the osimertinib, or pharmaceutically acceptable salt thereof, is administered to the human patient before the AZD2811 is administered to the human patient. In embodiments, the cancer is lung cancer, such as NSCLC. In yet further embodiments, the NSCLC is an EGFR mutation-positive NSCLC.

In an aspect there is provided an EGFR TKI for use in the treatment of cancer in a human patient, wherein the treatment comprises the separate, sequential, or simultaneous administration of i) the EGFR TKI and ii) AZD2811 to the human patient. In embodiments, the EGFR TKI is osimertinib or a pharmaceutically acceptable salt thereof. In further embodiments, the human patient is an EGFR TKI-naïve human patient. In further embodiments, the human patient has previously received EGFR TKI treatment. In further embodiments, the human patient has previously received osimertinib or a pharmaceutically acceptable salt thereof. In still further embodiments, the cancer is lung cancer, such as NSCLC. In yet further embodiments, the NSCLC is an EGFR mutation-positive NSCLC.

In an aspect there is provided a method of treating cancer in a human patient in need of such a treatment comprising the separate, sequential, or simultaneous administration of i) a therapeutically effective amount of an EGFR TKI and ii) a therapeutically effective amount of AZD2811 to the human patient. In embodiments, the EGFR TKI is osimertinib or a pharmaceutically acceptable salt thereof. In further embodiments, the human patient is an EGFR TKI-naïve human patient. In further embodiments, the human patient has previously received EGFR TKI treatment. In further embodiments, the human patient has previously received osimertinib or a pharmaceutically acceptable salt thereof. In still further embodiments, the cancer is lung cancer, such as NSCLC. In yet further embodiments, the NSCLC is an EGFR mutation-positive NSCLC.

In an aspect there is provided use of an EGFR TKI in the manufacture of a medicament for the treatment of cancer in a human patient, wherein the treatment comprises the separate, sequential, or simultaneous administration of i) the EGFR TKI and ii) AZD2811 to the human patient. In embodiments, the EGFR TKI is osimertinib or a pharmaceutically acceptable salt thereof. In further embodiments, the human patient is an EGFR TKI-naïve human patient. In further embodiments, the human patient has previously received EGFR TKI treatment. In further embodiments, the human patient has previously received osimertinib or a pharmaceutically acceptable salt thereof. In still further embodiments, the cancer is lung cancer, such as NSCLC. In yet further embodiments, the NSCLC is an EGFR mutation-positive NSCLC.

In an aspect there is provided AZD2811 for use in the treatment of cancer in a human patient, wherein the treatment comprises the separate, sequential, or simultaneous administration of i) an EGFR TKI and ii) the AZD2811 to the human patient. In embodiments, the EGFR TKI is osimertinib or a pharmaceutically acceptable salt thereof. In further embodiments, the human patient is an EGFR TKI-naïve human patient. In further embodiments, the human patient has previously received EGFR TKI treatment. In further embodiments, the human patient has previously received osimertinib or a pharmaceutically acceptable salt thereof. In still further embodiments, the cancer is lung cancer, such as NSCLC. In yet further embodiments, the NSCLC is an EGFR mutation-positive NSCLC.

In an aspect there is provided a method of treating cancer in a human patient in need of such a treatment comprising administration to the human patient a therapeutically effective amount of AZD2811, wherein the treatment comprises the separate, sequential, or simultaneous administration of i) a therapeutically effective amount of an EGFR TKI and ii) a therapeutically effective amount of the AZD2811 to the human patient. In embodiments, the EGFR TKI is osimertinib or a pharmaceutically acceptable salt thereof. In further embodiments, the human patient is an EGFR TKI-naïve human patient. In further embodiments, the human patient has previously received EGFR TKI treatment. In further embodiments, the human patient has previously received osimertinib or a pharmaceutically acceptable salt thereof. In still further embodiments, the cancer is lung cancer, such as NSCLC. In yet further embodiments, the NSCLC is an EGFR mutation-positive NSCLC.

In an aspect there is provided use of AZD2811 in the manufacture of a medicament for the treatment of cancer in a human patient, wherein the treatment comprises the separate, sequential, or simultaneous administration of i) an EGFR TKI and ii) the AZD2811 to the human patient. In embodiments, the EGFR TKI is osimertinib or a pharmaceutically acceptable salt thereof. In further embodiments, the human patient is an EGFR TKI-naïve human patient. In further embodiments, the human patient has previously received EGFR TKI treatment. In further embodiments, the human patient has previously received osimertinib or a pharmaceutically acceptable salt thereof. In still further embodiments, the cancer is lung cancer, such as NSCLC. In yet further embodiments, the NSCLC is an EGFR mutation-positive NSCLC.

In an aspect there is provided a kit comprising:

a first pharmaceutical composition comprising an EGFR TKI and a pharmaceutically acceptable carrier; and
a second pharmaceutical composition comprising AZD2811 and a pharmaceutically acceptable carrier.

In an aspect, there is provided AZD2811 for use in the treatment of non-small cell lung cancer in a human patient, wherein said patient's disease has progressed on or after previous EGFR TKI treatment. In embodiments, the human patient's disease has progressed on or after previous treatment with osimertinib, or a pharmaceutically acceptable salt thereof. In embodiments, said treatment with AZD2811 induces cell death in drug tolerant persister cells.

In an aspect, there is provided a method of treating non-small cell lung cancer in a human patient in need of such a treatment comprising administration to the human patient a therapeutically effective amount of AZD2811, wherein said patient's disease has progressed on or after previous EGFR TKI treatment. In embodiments, the human patient's disease has progressed on or after previous treatment with osimertinib, or a pharmaceutically acceptable salt thereof. In embodiments, said treatment with AZD2811 induces cell death in drug tolerant persister cells.

In an aspect, there is provided the use of AZD2811 in the manufacture of a medicament for the treatment of non-small cell lung cancer in a human patient, wherein said patient's disease has progressed on or after previous EGFR TKI treatment. In embodiments, the human patient's disease has progressed on or after previous treatment with osimertinib, or a pharmaceutically acceptable salt thereof. In embodiments, said treatment with AZD2811 induces cell death in drug tolerant persister cells.

### EXAMPLES

The specific Examples below, with reference to the accompanying Figures, are provided for illustrative purposes only and are not to be construed as limiting upon the teachings herein.

PC9 is a cell line derived from human lung adenocarcinoma harbouring the activating mutation in EGFR del E746_A750 (Ex19-del).

Unless otherwise stated, all reagents are commercially available and were used as supplied.

Example 1: A subset of osimertinib-resistant PC9 cell lines show increased sensitivity to Aurora B inhibition.

The purpose of this experiment was to show that a subset of osimertinib-resistant cell lines show enhanced sensitivity to an Aurora Kinase B inhibitor as well as enhanced sensitivity to an Aurora Kinase B inhibitor in combination with an EGFR TKI. The data demonstrate that this effect was achieved because decreased cell viability was observed in several osimertinib-resistant cell lines (PC9-AZDR1, PC9-AZDR2 and PC9-AZDR3) when treated with AZD2811 alone and AZD2811 in combination with osimertinib.

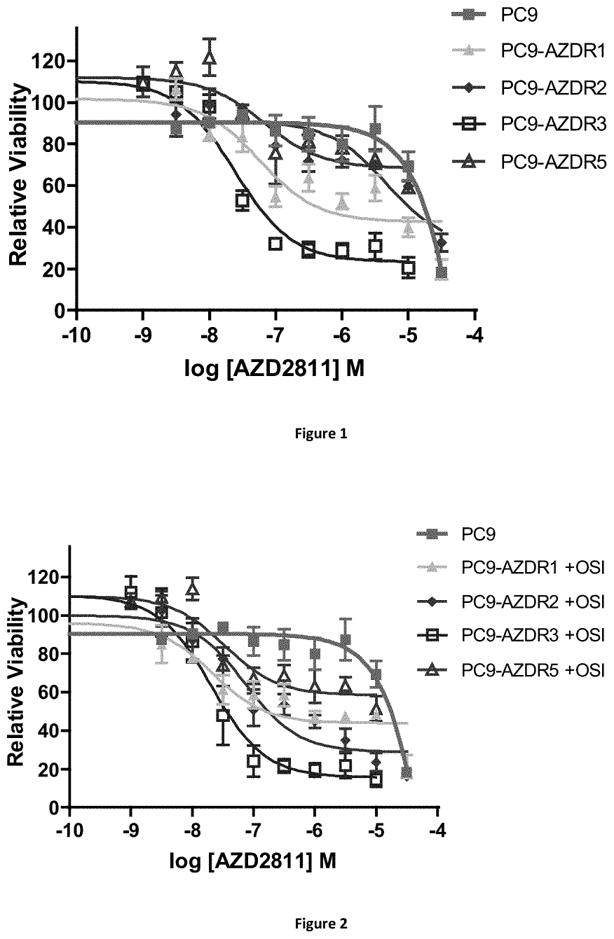

Dose response curves to AZD2811 were generated in parental PC9, as well as resistant lines PC9-AZDR1, PC9-AZDR2, PC9-AZDR3 and PC9-AZDR5. Resistant cells were generated by culturing parental PC9 cells in escalating doses of osimertinib (AZD9291) over time, up to a maximal dose of 1.5 μM (as described in Eberlein et al., Cancer Research 75(12):2489-500). Cells were treated with a 9-point dose escalation, from either 1 nm to 10 μM or 3 nM to 30 μM in half-log intervals, as well as vehicle (DMSO) control. Osimertinib-resistant cells were co-treated with 160 nM osimertinib (normal culture conditions) or drug-free control. After 96 h, cells were incubated with Cell Titer Glo reagent as per manufacturer's instructions, and luminescence was measured on a Tecan Magellan M200 plate reader. Data were normalized to DMSO control and plotted using PRISM software to generate IC50 values. The data are shown in FIGS. 1 and 2.

| | AZD2811 | AZD2811 |
|---|---|---|
| | IC50 (nM) no | IC50 (nM) + |
| Cell line | osimertinib | osimertinib |
| PC9 | >10000 | N/A |
| PC9-AZDR1 | 399 | 159 |
| PC9-AZDR2 | >10000 | 173 |
| PC9-AZDR3 | 55 | 36 |
| PC9-AZDR5 | >10000 | >10000 |

Example 2. Osimertinib-resistant cell lines that show decreased viability after Aurora inhibition undergo significant apoptosis at doses that do not affect parental cells.

The purpose of this experiment was to show that osimertinib-resistant cell lines that show enhanced sensitivity to Aurora Kinase B inhibition undergo apoptosis in response to drug. The data demonstrate that this effect was achieved because an osimertinib resistant cell line (AZDR3) showed enhanced caspase activity (an indicator of apoptosis) when treated with AZD2811 when compared to a parental cell line (PC9) treated in the same way.

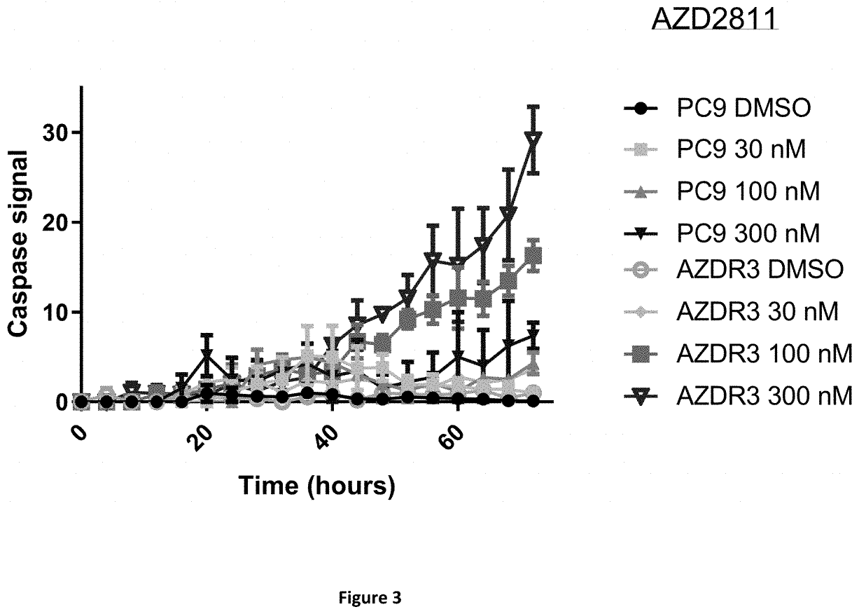

PC9 parental cells and PC9-AZDR3 resistant cells were seeded in 96 well plated at a concentration of 5000 cells/well. The next day, cells were treated with the doses of AZD2811 (30 nM, 100 nM and 300 nM; n=2), as well as the Incucyte Caspase 3/7 reagent (green) at a final concentration of 1 μM. Cells were then placed on the Incucyte S3 imaging system and both cell confluence and green fluorescence were measured, every 4 hours. After 72 h the experiment was terminated, and arbitrary apoptosis values calculated by dividing the number of individual green points (apoptosis events) by cell confluence. For each cell line, data was normalized to DMSO treated values at 72 h. The data are shown in FIG. 3.

Example 3. AZD2811 treatment inhibits the establishment of drug-tolerant persister cells after osimertinib treatment and inhibits the re-growth of persister cells after osimertinib monotherapy.

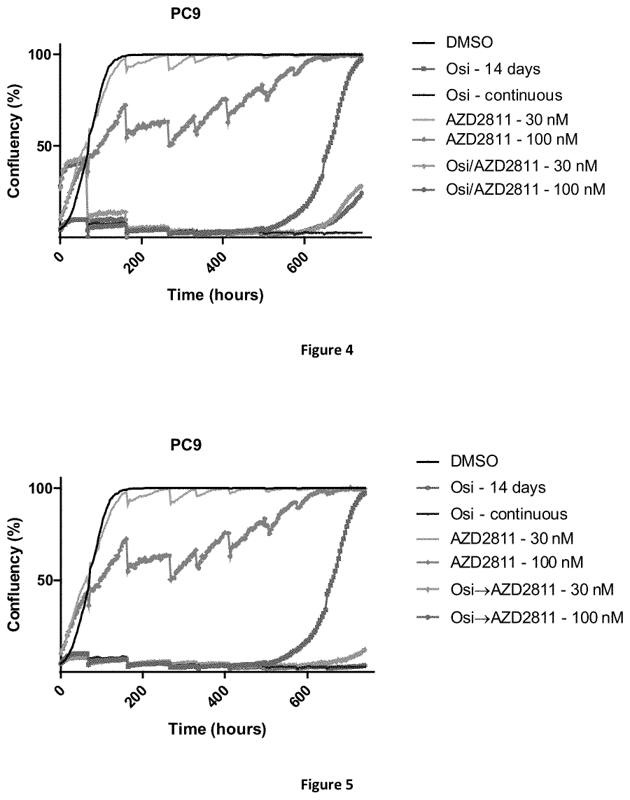

The purpose of this experiment was to show that treatment with an Aurora Kinase B inhibitor inhibits the establishment of drug-tolerant persister cells after EGFR TKI treatment and inhibits the re-growth of persister cells after EGFR TKI monotherapy. The data demonstrate that this effect was achieved because cells treated for 14 days with a combination of osimertinib and AZD2811 showed a lower percentage confluency (a measure of cell growth) at 31 days than cells treated for 14 days with osimertinib alone (FIG. 4). Similarly, cells treated for 14 days with osimertinib followed by 17 days treatment with AZD2811 showed a lower percentage confluency (a measure of cell growth) at 31 days than cells treated for 14 days with osimertinib alone without subsequent treatment with AZD2811 (FIG. 5).

PC9 cells were plated in 48 well plates at a concentration of 40,000 cells/well. The following day cells were treated with either osimertinib monotherapy (500 nM), doses of AZD2811 (30 nM and 100 nM), or the combination of the two agents, and confluence measurement was begun using the Incucyte imaging platform. After 14 days, combination treated wells, as well as one subset of osimertinib monotherapy wells, were washed 2× with phosphate-buffered saline (PBS) and replaced with drug-free media. Further subsets of osimertinib monotherapy-treated wells were washed 2× with PBS and replaced with media containing the indicated doses of AZD2811 monotherapy. Confluence measurements continued for a further 17 days, and results were plotted using PRISM software. The data are shown in FIGS. 4 and 5.

Example 4. AZD2811 treatment induces apoptosis in osimertinib drug-tolerant persister cells and enhances osimertinib-induced apoptosis in PC9 cells.

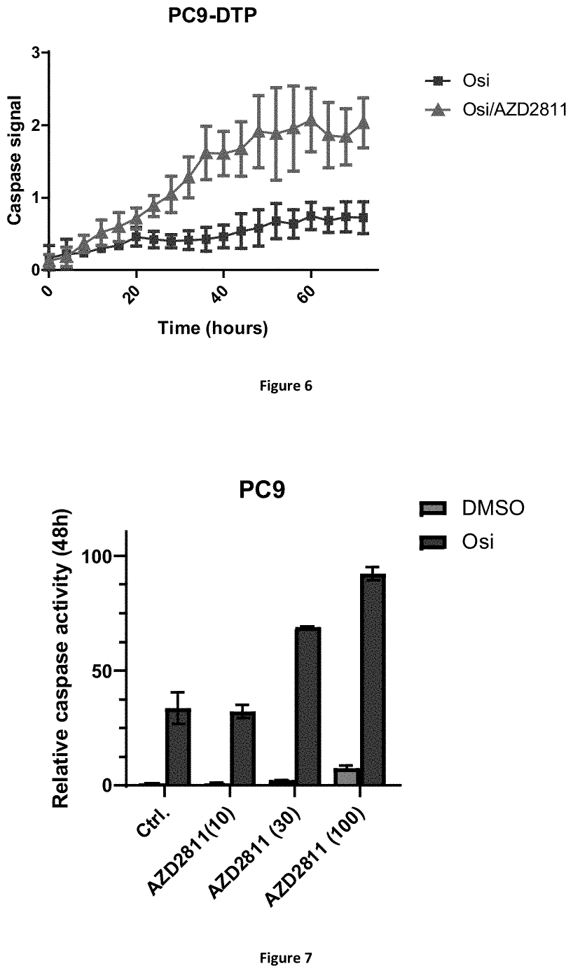

The purpose of this experiment was to show that treatment with an Aurora Kinase B inhibitor induces apoptosis in osimertinib drug-tolerant persister (DTP) cells and enhances EGFR TKI-induced apoptosis in PC9 cells. The data demonstrate that this effect was achieved because enhanced caspase activity (an indicator of apoptosis) was observed in DTP cells treated with a combination of osimertinib and AZD2811 when compared to DTP cells treated with osimertinib alone (FIG. 6). In addition, enhanced caspase activity (an indicator of apoptosis) was observed in PC9 cells treated with a combination of osimertinib and AZD2811 when compared to PC9 cells treated with osimertinib alone (FIG. 7).

A) PC9 parental cells were treated for 14 days with 500 nM osimertinib to establish drug-tolerant persister cells. At this time, cells were treated with the osimertinib (500 nM)+AZD2811 (100 nM) combination, or continued osimertinib monotherapy (500 nM), along with Incucyte Caspase 3/7 reagent (1 μM). Cells were then placed on the Incucyte S3 imaging system and both cell confluence and green fluorescence were measured, every 4 hours. After 72 h the experiment was terminated, and arbitrary apoptosis values calculated by dividing the number of individual green points (apoptosis events) by cell confluence. For treatment, data was normalized to osimertinib monotherapy treated values at 72 h. The data are shown in FIG. 6.

B) PC9 parental cells were treated with either osimertinib (500 nM), AZD2811 (10, 30 or 100 nM) or their combination, along with Incucyte Caspase 3/7 reagent (1 μM). After 48 h, cells were placed on the Incucyte S3 imaging system, and analysed as above. Data was normalized to values calculated for DMSO control. The data are shown in FIG. 7.

Example 5: AZD2811 combined with osimertinib prevents the regrowth of PC9 xenograft tumours compared to osimertinib monotherapy.

The purpose of this experiment was to show that an Aurora Kinase B inhibitor in combination with an EGFR TKI is more effective at preventing regrowth of PC9 xenograft tumours than EGFR TKI monotherapy. In the experiment, PC9 xenograft tumours were treated separately with osimertinib monotherapy (28 days) and a combination of osimertinib and AZD2811 (28 days). The data demonstrate that the combination treatment was more effective than osimertinib monotherapy because tumour regrowth was observed in PC9 xenograft tumours treated with monotherapy at around 80 days, whereas PC9 xenograft tumours treated with combination therapy remained progression-free for at least 220 days.

PC9 xenograft tumours were established by subcutaneous implantation of 5×10⁶ cells per animal, in 100 μl of cell suspension including 50% matrigel, into the dorsal left flank of female SCID mice. All mice were older than 6 weeks at the time of cell implant. Tumor growth was monitored twice weekly by bilateral calliper measurements and tumour volume calculated using elliptical formula (pi/6×width×width×length).

Mice were randomized into vehicle or treatment groups with approximate mean start size of 0.2 cm3. Randomization for animal studies was based on initial tumour volumes to ensure equal distribution across groups. Mice were dosed daily by oral gavage with vehicle or 25 mg/kg osimertinib or once weekly by intravenous with 25 mg/kg AZD2811 or with the combination of osimertinib and AZD2811. Note that all treatments were stopped on day 28. However, tumour growth was followed for approximately 200 additional days.

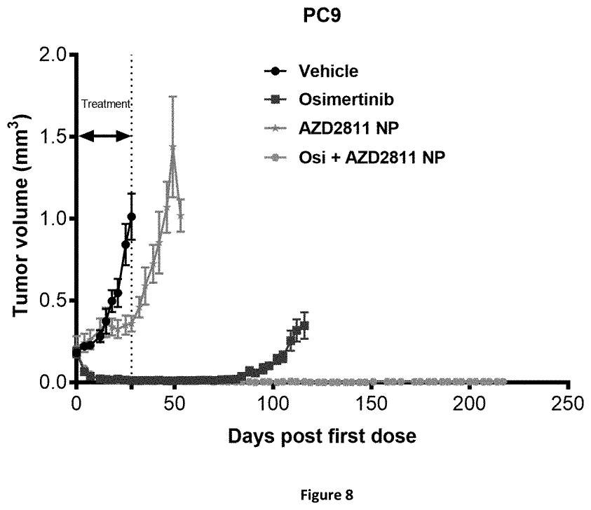

The data in FIG. 8 show that a 4-week long treatment with osimertinib monotherapy at 25 mg/kg resulted in complete shrinkage but tumours reappeared shortly after oral administrations were stopped. AZD2811 monotherapy caused only limited tumour shrinkage, but when it was combined with osimertinib clear cooperative effects were observed, with PC9 xenografts remaining progression-free for at least 220 days.

1-13. (canceled)

14. A method of treating cancer in a human patient in need of such a treatment comprising the administration to the human patient of a therapeutically effective amount of an EGFR TKI, wherein the EGFR TKI is administered in combination with a therapeutically effective amount of AZD2811.

15. The method according to claim 14, wherein the EGFR TKI is a compound of Formula (I): 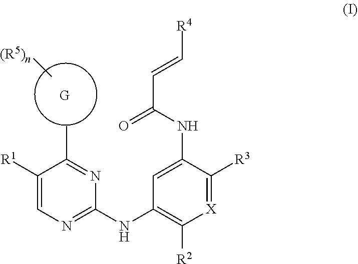
wherein:
G is selected from 4,5,6,7-tetrahydropyrazolo[1,5-a]pyridin-3-yl, indol-3-yl, indazol-1-yl, 3,4-dihydro-1H-[1,4]oxazino[4,3-a]indol-10-yl, 6,7,8,9-tetrahydropyrido[1,2-a]indol-10-yl, 5,6-dihydro-4H-pyrrolo[3,2,1-ij]quinolin-1-yl, pyrrolo[3,2-b]pyridin-3-yl and pyrazolo[1,5-a]pyridin-3-yl;
R¹ is selected from hydrogen, fluoro, chloro, methyl and cyano;
R² is selected from methoxy, trifluoromethoxy, ethoxy, 2,2,2-trifluoroethoxy and methyl;
R³ is selected from (3R)-3-(dimethylamino)pyrrolidin-1-yl, (3S)-3-(dimethyl-amino)pyrrolidin-1-yl, 3-(dimethylamino)azetidin-1-yl, [2-(dimethylamino)ethyl]-(methyl)amino, [2-(methylamino)ethyl](methyl)amino, 2-(dimethylamino)ethoxy, 2-(methylamino)ethoxy, 5-methyl-2,5-diazaspiro[3.4]oct-2-yl, (3aR,6aR)-5-methylhexa-hydro-pyrrolo[3,4-b]pyrrol-1(2H)-yl, 1-methyl-1,2,3,6-tetrahydropyridin-4-yl, 4-methylpiperizin-1-yl, 4-[2-(dimethylamino)-2-oxoethyl]piperazin-1-yl, methyl[2-(4-methylpiperazin-1-yl)ethyl]amino, methyl[2-(morpholin-4-yl)ethyl]amino, 1-amino-1,2,3,6-tetrahydropyridin-4-yl and 4-[(2S)-2-aminopropanoyl]piperazin-1-yl;
R⁴ is selected from hydrogen, 1-piperidinomethyl and N,N-dimethylaminomethyl;
R⁵ is independently selected from methyl, ethyl, propyl, 2,2-difluoroethyl, 2,2,2-trifluoroethyl, fluoro, chloro and cyclopropyl;
X is CH or N; and
n is 0, 1 or 2;
or a pharmaceutically acceptable salt thereof.

16. The method according to claim 14, wherein the EGFR TKI is selected from the group consisting of osimertinib or a pharmaceutically acceptable salt thereof, AZD3759 or a pharmaceutically acceptable salt thereof, lazertinib or a pharmaceutically acceptable salt thereof, abivertinib or a pharmaceutically acceptable salt thereof, alflutinib or a pharmaceutically acceptable salt thereof, afatinib or a pharmaceutically acceptable salt thereof, CX-101 or a pharmaceutically acceptable salt thereof, HS-10296 or a pharmaceutically acceptable salt thereof, BPI-7711 or a pharmaceutically acceptable salt thereof, dacomitinib or a pharmaceutically acceptable salt thereof, icotinib or a pharmaceutically acceptable salt thereof, gefitinib or a pharmaceutically acceptable salt thereof and erlotinib or a pharmaceutically acceptable salt thereof.

17. The method according to claim 14, wherein the EGFR TKI is selected from the group consisting of osimertinib or a pharmaceutically acceptable salt thereof, AZD3759 or a pharmaceutically acceptable salt thereof, HS-10296 or a pharmaceutically acceptable salt thereof, and lazertinib or a pharmaceutically acceptable salt thereof.

18. The method according to claim 14, wherein the EGFR TKI is osimertinib or a pharmaceutically acceptable salt thereof.

19. The method according to claim 14, wherein the cancer is lung cancer.

20. The method according to claim 19, wherein the cancer is non-small cell lung cancer (NSCLC).

21. The method according to claim 20, wherein the NSCLC is an EGFR mutation-positive NSCLC.

22. The method according to claim 21, wherein the EGFR mutation-positive NSCLC comprises activating mutations in EGFR selected from exon 19 deletions or L858R substitution mutations.

23. The method according to claim 21, wherein the EGFR mutation-positive NSCLC comprises the T790M mutation.

24. The method according to claim 14, wherein the human patient is an EGFR TKI-naïve human patient.

25. The method according to claim 14, wherein the human patient's disease has progressed on or after previous EGFR TKI treatment.

26. The method according to claim 14, wherein the human patient's disease has progressed on or after previous treatment with osimertinib, or a pharmaceutically acceptable salt thereof.
